(19) United States
(12) Patent Application Publication
(10) Pub. No.: US 2006/0287693 A1
(43) Pub. Date: Dec. 21, 2006

(54) Implanted telephone system

(76) Inventors: Clifford Kraft, Naperville, IL (US); Vasilios D. Dossas, Chicago, IL (US)

Correspondence Address: Clifford Kraft, 320 Robin Hill Dr., Naperville, IL, 60540, US

(21) Appl. No.: 11/446,519

(22) Filed: Jun. 2, 2006

Related U.S. Application Data
(60) Provisional application No. 60/688,442, filed on Jun. 8, 2005.

(51) Int. Cl. A61N 1/00

(52) U.S. Cl. 607/60

(57) ABSTRACT

An wireless telephone microchip can be implanted into a human or animal to allow telephone calling, location and control of implanted medical devices. A chip can contain complete telephone circuitry, GPS and local RF communication to an earphone/microphone for telephony and for controlling or modifying a medical device. Wideband local RF can provide downloaded compressed music to a high quality earphone. The chip could optionally contain a small microphone and earphone that operates through the skin. The chip can contain a battery that can be recharged through an externally applied field or by other means. In particular, the battery could be recharged while the user sleeps in a bed containing an induction coil.

This application relates to and claims priority from United States Provisional patent application number 60/688,442 filed Jun. 8, 2005. Application 60/688,442 is hereby incorporated by reference.

### BACKGROUND

1. Field of the invention

The present invention relates generally to wireless communication and more particularly to a telephone that is implanted in a human or animal body that can be used for normal telephone communication, location determination, the controlling of the administration of drugs and for other uses.

2. Description of the Prior Art

Prior art radio communications devices have been implanted into animals for location and identification. In particular tags known as RFID tagbs have been implanted in animals to identify them upon scanning with a wand. An RFID type chip has been developed by a company called Applied Digital Solutions that can be implanted in a human. Chips like these have been used in Europe and elsewhere to identify people, allow entry into clubs and other locations control bar-tabs and other uses. These chips can only be read out at close range. RF transponders also have been attached, and in some cases, implanted in animals to allow location of these animals in their natural habitats.

Location of missing pets or children is a problem that has major interest in the U.S. and elsewhere. In particular, there have been numerous kidnappings and disappearances of children in the U.S. and other countries in recent years. In addition, children sometimes wander off or get lost at shopping malls, on camping trips and various other places. Pets run away or slip out of houses or otherwise get lost. Currently manufactured cellular telephones are equipped with features that allow accurate GPS location upon request from a telephone service provider. Companies such as Qualcomm make complete telephone/GPS chip sets for this purpose. GPS cellular telephones could help partially solve this problem.

There is also a continually growing market for implanted medical devices such as pacemakers and drug delivery systems (insulin for example). These devices usually run unattended within the body. Some of them can have parameters changed by close-range communication with a wand or communications device. However, there is no really good way to control them or update them from a distance.

The cellular telephone has become ubiquitous throughout developed society, especially in the U.S., Europe and Japan. Some people spend numerous hours communicating on the cellular telephones. It would be advantageous to have a cellular telephone that could be implanted in a human or animal body if this telephone could be used for normal telephone voice communication, data communication, optional location and optional control of implanted medical devices.

### SUMMARY OF THE INVENTION

The present invention relates to a communications apparatus including a telephone circuit implanted in a human or animal body with the telephone circuit being powered from a battery also implanted or external. The telephone circuit can optionally communicate locally with an earphone and microphone preferably using local wireless communications such as RF, infrared, ultrasound or by any other local communications technique.

The implanted chip can also contain a GPS receiver that can optionally be of the assisted type to allow tracking or finding of the person or animal implanted.

The telephone circuit of the implanted chip can optionally communicate locally with an implanted medical device to control it or modify it.

The telephone circuit can optionally include an earphone and/or microphone operating through human skin. This would allow a telephone mounted under the skin on the back of the wrist to operate exactly as a wrist radio.

### DESCRIPTION OF THE DRAWINGS

FIG. 1 shows a telephone chip implanted in a human arm. The chip can communicate locally with an earphone/microphone and an implanted medical device.

FIGS. 2A-2B show a top and a side view of a possible implantable telephone chip with an antenna and battery.

FIG. 3 shows the use of an array of patch antenna elements.

FIG. 4 shows a block diagram of an implantable telephone chip.

FIG. 5 shows schematically a method to recharge the battery of an implanted chip.

FIG. 6 shows the method of FIG. 4 in a bed.

FIG. 7 shows an alternative method of recharging the battery in an implanted chip.

Several illustrations and drawings have been presented to better aid in the understanding of the present invention. The scope of the present invention is not limited to the figures.

### DESCRIPTION OF THE INVENTION

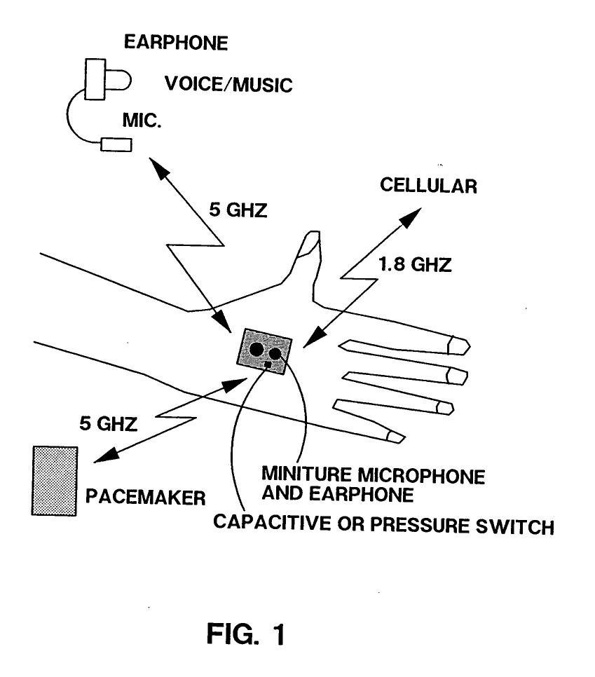

The present invention relates to an implanted radio chip preferably a cellular telephone chip. Turning to FIG. 1, a chip can be seen implanted in a human hand. The chip or chips of the present invention can be implanted anywhere in a human or animal body. The chip shown in FIG. 1 can be self-contained by having a battery power supply, a cellular or other radio transmitter/receiver and optionally several secondary radio transmitters, receivers or transceivers.

The chip shown in FIG. 1 can, in a preferred embodiment, can provide cellular communications in the 1.8 MHz band (or alternatively the 800 MHz or other band), secondary communication with a earphone/microphone and secondary communication with an implanted medical device such as a pacemaker. In this embodiment, secondary communication can take place around 5 GHz or other frequencies such as an ISM band. An example of secondary communication that is within the scope of the present invention is the method known as Bluetooth.

In an alternative embodiment, the chip could contain a miniature microphone and/or earphone that transmits sound through a layer of skin. In this embodiment, the present invention can function as a wrist-radio where the user can speak directly into the chip and hold the chip to the ear to hear. In addition, an optional capacitive or pressure button could be used to activate calls. Requiring a code or a number of successive pushes (like 5 successive pushes in 2 seconds) would prevent the accidental pushing of the button from initiating calls. Calls could also be initiated by a voice recognition circuit attached to the microphone (recognition could be very simple by simply recognizing one or two words). Such a circuit could be used to “wake-up” the telephone from a power-saver mode on a voice command.

In addition to communicating with an earphone and microphone, secondary communication can also control a vibrator, ringer, call initiate button, keyboard/keypad, display, or other devices using short-range wireless communication techniques. For example, a user of an implanted cellular telephone wishing to make a voice call, could push a tiny button on the earphone to signal the telephone chip to initiate the call (via a secondary channel). A voice prompt in the earphone could tell the user to speak a telephone number, a name or a speed dial digit. For example, the user could say the command “Call Paul”. The telephone chip could then access a stored library of names, find a telephone number and place the call. When the call was finished, the chip could hang up automatically, or the user could again push the button.

In a location application, the present invention can be used in conjunction with a GPS receiver circuit using regular GPS or assisted GPS as is known in the art. When used for location, one application of the present invention can be to foil kidnappers (of children or pets). For this reason, an embodiment of the invention may implement a detector to detect any removal of the chip from the body site. This could prevent a kidnapper from simply cutting the chip out with a knife or other means. Upon detected removal from the body, the chip could send out an emergency alert signal to a unit owned by the parent that will record the last known location and optionally call 911, E911, or could send a message to a special service bureau providing location services. The simplest detector could be a thermal detector that senses body temperature.

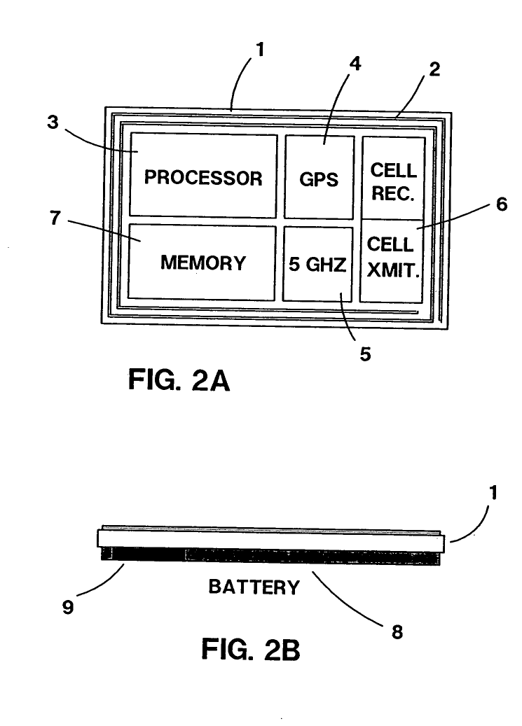

The present invention can generally be made as a silicon chip 1 as shown in FIG. 2A. In a preferred embodiment, a silicon chip (or group of chips) or other substrate or substrate material can contain a processor 3 a memory 7, a GPS receiver 4 a cellular transceiver 6 and at least one secondary transceiver 5 at 5 GHz or any other frequency. A dipole antenna 2, or other antenna, can be threaded around the chip or provided in any other way.

A preferred embodiment for the implanted chip of the present invention is a processor 3 such as those found in cellular telephones known in the art (but preferably smaller), an RF section of a cellular telephone 6 with assisted GPS and Bluetooth on a small semiconductor chip (of silicon, gallium-arsenide or any other material). This chip could be attached above or below a flat battery 8 as shown in FIG. 2B. The battery 8 can have a charger 9 as will be described. The battery could also be located externally to the chip.

It is known that RF transmission effectiveness in a device like that shown in FIGS. 2A-2b generally depends on transmit power antenna physical size. The optimum antenna length for an 1800-1900 MHz telephone of the type known today is around 4 cm (using ¼ wavelength at 1800 MHz). It is known in the art to reduce this length by using real and artificial inductive loading and other techniques. Artificial inductive loading can be optionally achieved using phase-shift transistors rather than physical inductors to create currents that lag voltages by 90 degrees. These artificial inductors can exhibit all properties of high-Q magnetic inductors and can be used for antenna loading coils. With a 1 cm square chip, an antenna could be laid around the perimeter making about 4 cm per revolution. The same antenna can optionally be shared between the GPS receiver, the secondary transceiver and the telephone transceiver using diplexing or antenna multiplexing techniques known in the art.

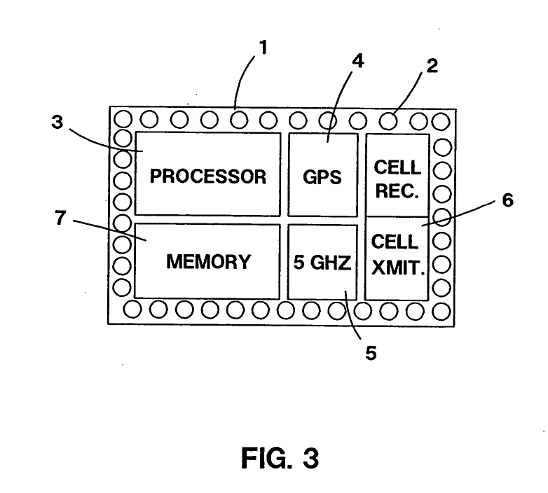

FIG. 3 shows an alternative antenna method using one or more patch antennas 2. The patch antennas shown in FIG. 3 are phased to form an array with the around the same physical capture area of a dipole antenna. Each patch could be driven at a slightly different phase to produce an optimum transmit or receive pattern. Phase shifting can be capacitive or inductive as is known in the art with electronic inductors as described above.

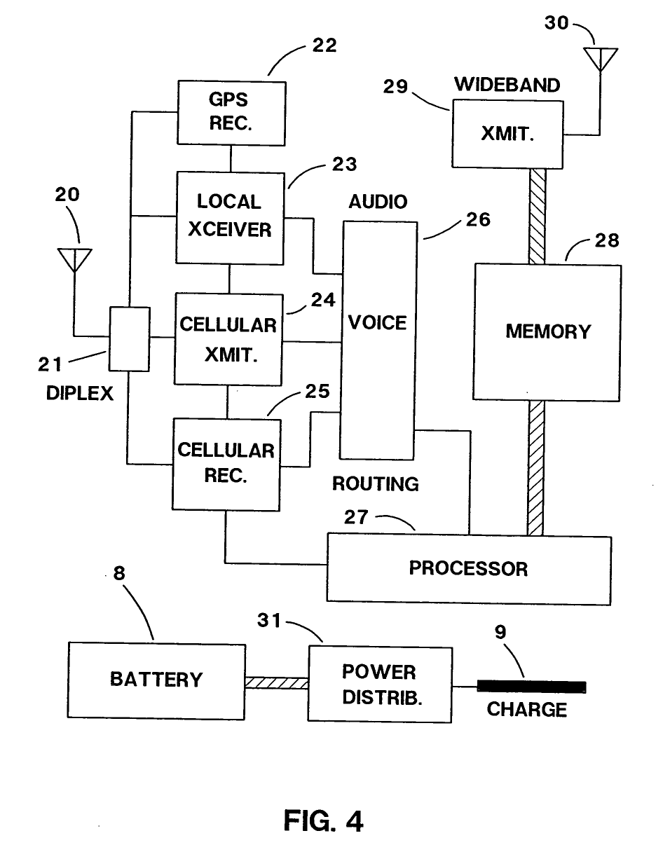

Turning to FIG. 4, a block diagram of an embodiment of the present invention is seen. A processor 27 coupled to a memory 28 can be seen controlling several RF units including a cellular transmitter 24, a cellular receiver 25, a local transceiver 23, a GPS receiver 22 and a wideband transmitter 29. The cellular transmitter and receiver can be connected to a cellular antenna 20 by means of a diplexer 21.

The cellular transmitter and receiver 25, 24 can be typical, albeit very small, cellular RF units adapted for use in cellular telephone frequency bands such as 1800 MHz or 800 MHz. They can also be any type of radio units. The local transceiver 23 can be a very low power unit running on an ISM band such as according to the Bluetooth standard or can be a specialized, or other, radio unit running on any frequency. The 5 GHz bands (and higher) are attractive because of the small antenna sizes normally used. The GPS receiver 22 can be of the type manufactured by Qualcomm and others that receive signals from GPS satellites. The processor can be an ARM unit or other processor normally used with cellular telephones. It can be any other type of processor including a specialized processor. The memory 28, while drawn in FIG. 4 as a single unit, can be several units of the same or different types such as RAM, DRAM, Flash, EEProm and the like. The memory 28 can also include long-term storage such as a miniature disk unit, memory cube, quantum memory or any other type of memory device or devices.

In FIG. 4, a audio voice routing module 26 can be seen controlled by the processor 27 that can route voice signals to and from the local transceiver 23 and the cellular RF units 24, 25. This voice router 26 can take demodulated incoming telephone audio from the cellular receiver 25 and send it to an earphone via local RF. It can also receive audio from a microphone via local RF and route it to the cellular transmitter 24.

Music can optionally be downloaded in compressed form from the internet via the cellular telephone and then played back through a high quality earphone (Hi-Fi) by means of storing the compressed audio in a part of the memory 28 and then transferring it to the high quality earphone by means of an optional wideband transmitter 29 shown with an optional second antenna 30. The antennas 20 and 30 can be the same antenna in some embodiments of the present invention. The music can be compressed by any means, or according to any method, known in the art. The preferred way is according to the MP3 standard.

A battery 8 and battery charger 9 can also be seen in FIG. 4. Power from the battery 8 can be distributed and controlled by a power distribution circuit 31. In an implanted chip, battery power conservation becomes very important. The implanted telephone of the present invention could conserve power by entering a “sleep” mode when not being actively used. Such a sleep mode might only allow circuitry to operate that receives incoming calls. A further, deeper “sleep” mode might simply shut the telephone off completely or almost completely. Choice of these various operational modes could be made by either a button on the chip itself, a button or control on an earphone or other external module or by voice recognition. Any method of controlling operational modes is within the scope of the present invention.

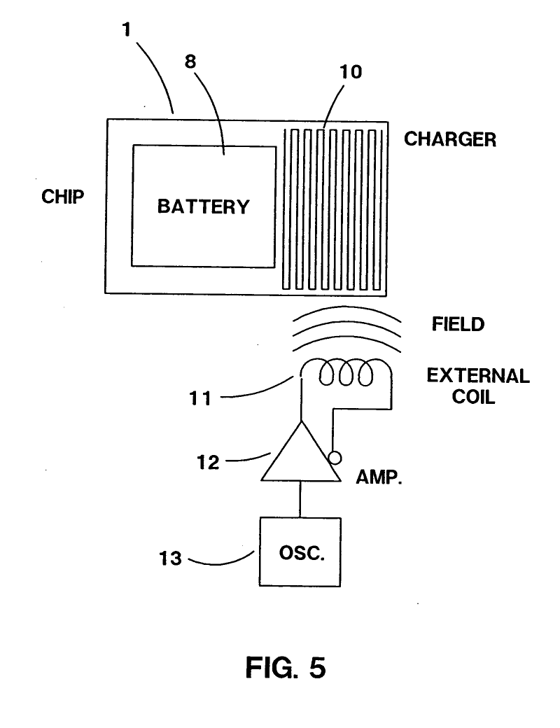

FIG. 5 shows a view of an embodiment of the bottom of an implanted chip 1. A battery 8 and charger receiving coil 10 can be seen. An external circuit can include an oscillator 13, an amplifier 12 and an induction coil 11. The oscillator 13 normally produces alternating current (AC) signals preferably in the frequency range of from 1-10 kHz. While this frequency range is preferred, any AC frequency is within the scope of the present invention. The amplified current is driven through the induction coil 11 to produce an external alternating magnetic field. This field can be coupled into the receiving coil 10 of the chip 1 to produce a voltage in a transformer action. After rectification, the resulting current can be used to charge the battery 8.

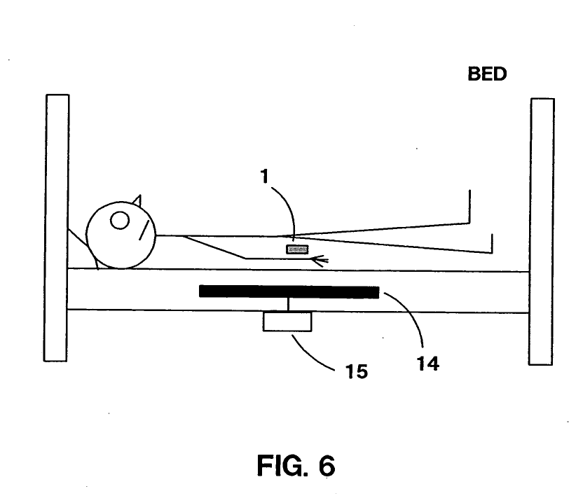

FIG. 6 shows how the principle shown in FIG. 5 can be used to charge an implanted chip 1 in a person's arm while the person sleeps. The external part of the circuit of FIG. 5 can be contained in a control unit 15 in the vicinity of the bed. An large induction coil 14 can produce the necessary magnetic field. Transformer action causes the receiving coil in the chip to generate a voltage which drives a current into the battery so that the battery charges.

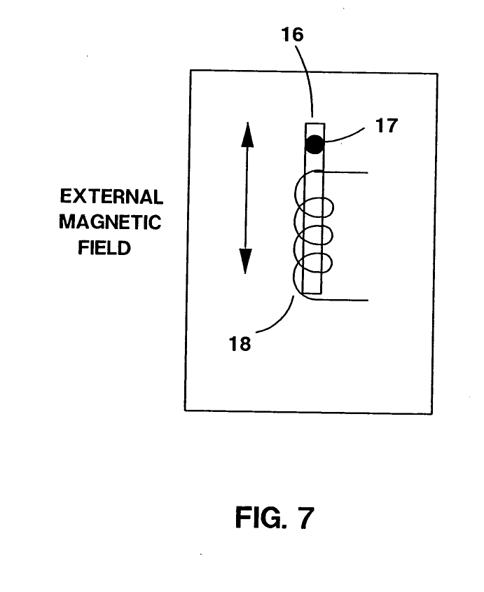

FIG. 7 shows an mechanical battery charger that could be optionally used in an implanted chip. Here a small tube 16 can contain a magnetized metal ball 17 that is free to move back and forth in the tube as the user moves (for example for a chip implanted in the arm, as the user moves his arm). The motion of the magnetic ball 17 causes a charging voltage to be induced in the coil 18 which can drive a charging current into the battery to charge it. Any method of charging the battery in the implanted telephone chip is within the scope of the present invention.

Several descriptions and illustrations have been presented to better aid in the understanding of the present invention. A person skilled in the art will realized that many changes and variations are possible. All of these changes and variations are within the scope of the present invention.

We claim:

1. A communications apparatus comprising a telephone circuit implanted in a human or animal body, said telephone circuit powered from a battery.

2. The communications apparatus of claim 1 wherein said telephone circuit communicates locally with an earphone and microphone.

3. The communications apparatus of claim 2 wherein said apparatus communicates locally using wireless communications.

4. The communications apparatus of claim 1 further comprising a GPS receiver.

5. The communications apparatus of claim 1 wherein said telephone circuit communicates locally with an implanted medical device.

6. The communications apparatus of claim 1 wherein said telephone circuit includes an earphone and microphone operating through human skin.

7. The communications apparatus of claim 1 wherein said battery is also implanted.

8. The communications apparatus of claim 7 wherein said battery is adjacent to said telephone circuit.

9. An cellular telephone circuit implanted in a human or animal body comprising, in combination:
a cellular telephone RF transceiver;
a cellular telephone antenna;
a local communications transceiver;
an audio circuit;
said cellular telephone RF transceiver modulating audio signals from said audio circuit to produce modulated cellular telephone radio signals to said cellular telephone antenna and demodulating cellular telephone radio signals from said cellular telephone antenna to produce audio signals to said audio circuit;
said local communications transceiver relaying said audio signals to and from a remote earphone/microphone device.

10. A method of providing telephone communication to an individual comprising the steps of:
implanting a telephone circuit in said individual's body;
powering said telephone circuit with a battery also implanted in said individual's body.

11. The method of claim 10 further comprising implanting a GPS receiver with said telephone circuit.

12. The method of claim 10 further comprising charging said battery using a charger external to said individual.

13. The method of claim 12 wherein charging said battery is performed using a time-varying magnetic field.

14. The method of claim 10 further comprising said telephone circuit communicating wirelessly with an earphone and microphone.

15. The method of claim 10 further comprising said telephone circuit communicating locally with a medical device implanted in said individual.

16. The method of claim 15 wherein said telephone circuit communicates wirelessly with said medical device.

17. The method of claim 10 wherein said implanted telephone contains an earphone and microphone operating through human skin.
